(19) United States
(12) Patent Application Publication
(10) Pub. No.: US 2026/0283930 A1
(43) Pub. Date: Sep. 24, 2026

(54) COMPOSITION WITH CHITOSAN AND NANOMETRIC SPECIFIC SURFACE TREATED-MINERAL PIGMENT

(71) Applicant: L'OREAL, Paris (FR)

(72) Inventors: Danielle LE VERGE, Chevilly Larue (FR); Toni MOINE, Chevilly Larue (FR); Christophe KUSINA, Chevilly Larue (FR)

(73) Assignee: L'OREAL, Paris (FR)

(21) Appl. No.: 19/473,517

(22) PCT Filed: Apr. 18, 2024
(86) PCT No.: PCT/EP2024/060536

§ 371 Date: Oct. 8, 2025

(30) Foreign Application Priority Data
Apr. 18, 2023 (EP) 23305590.4

(51) Int. Cl. A61K 8/73; A61K 8/29; A61Q 1/02

(52) U.S. Cl. CPC A61K 8/736; A61K 8/29; A61Q 1/02; A61K 2800/34; A61K 2800/413; A61K 2800/43; A61K 2800/612; A61K 2800/651

(57) ABSTRACT

The present invention relates to a cosmetic composition comprising, in a physiologically acceptable aqueous medium:
a) at least 0.01% by weight with respect to the total weight of the composition of native chitosan having a molecular weight strictly greater than 3000 Dalton, this quantity being strictly less than 15% by weight, and
b) at least one surface-treated mineral pigment, said pigment having a size less than or equal to 100 nm.

It also relates to a process for make-up and/or care of the skin and/or skin appendages, wherein the composition according to the invention is applied on the skin and/or skin appendages.

The present invention relates to a cosmetic composition comprising, in a physiologically acceptable aqueous medium:
a) at least 0.01% by weight with respect to the total weight of the composition of native chitosan having a molecular weight strictly greater than 3000 Dalton, this quantity being strictly less than 15% by weight, and
b) at least one surface-treated mineral pigment, said pigment having a size less than or equal to 100 nm.

It also relates to a process for make-up and/or care of the skin and/or skin appendages, wherein the composition according to the invention is applied on the skin and/or skin appendages.

There are many cosmetic compositions for which resistance properties of the deposited film, after application on keratin materials, are desired. Mention may be made for example of lipsticks or nail varnishes. In order to obtain such a result, it is possible to combine particular raw materials, in particular film-forming agents. However, the presence of such agents for the resistance of the film on keratin materials can lead to compositions that cause dryness. Moreover, the film-forming agents commonly used are typically petrochemical in nature, such as silicones or acrylic polymers.

Moreover, it is often sought to obtain compositions with good covering power.

The formulator is therefore seeking raw materials and/or systems making it possible to obtain fluid (i.e., flowing) compositions, the deposit whereof is covering, homogeneous and resistant (i.e., having a good resistance), in particular having a good resistance to water.

Moreover, the formulation of environmentally friendly solutions, the design and development of which account for environmental challenges is becoming a major concern to help meet global challenges.

It has therefore become essential to propose more sustainable compositions and/or preparation processes and/or ingredients to meet these environmental challenges.

In this context, it is important to develop novel cosmetic compositions with a better carbon footprint, in particular by promoting the use of renewable raw materials and/or those with a good wilderness quality index and/or of natural origin, and more particularly those of plant origin, while reducing the use of compounds of petrochemical origin.

The aim of the present invention is that of providing aqueous cosmetic compositions having good make-up properties, in particular in terms of covering power and homogeneity, but also having a good resistance, in particular having a good resistance to water. After application, these compositions leave a covering and homogeneous film-forming deposit, which has a good resistance to wear. The films formed are adhesive and cohesive, and have an enhanced resistance to water.

Moreover, the surface treatment of the mineral pigment, said pigment having a size less than or equal to 100 nm, makes it possible to enhance the sun protection factor of the composition obtained.

These compositions also comprise sustainable ingredients, thus making it possible to meet environmental challenges.

The present invention therefore relates to a cosmetic composition, in particular for make-up and/or care of the skin and/or lips, in particular for skin care, comprising, in a physiologically acceptable aqueous medium:
a) at least 0.01% by weight with respect to the total weight of the composition of native chitosan having a molecular weight strictly greater than 3000 Dalton, this quantity being strictly less than 15% by weight, and
b) at least one surface-treated mineral pigment, said pigment having a size less than or equal to 100 nm.

“Physiologically acceptable” refers to a medium compatible with keratin materials.

It also relates to a process for make-up and/or care of the skin and/or skin appendages, wherein the composition according to the invention is applied on the skin and/or skin appendages.

Preferably, the composition according to the invention is substantially free from (meth)acrylic polymer. Preferably, the composition according to the invention is substantially free from silicone.

“Substantially free from (meth)acrylic polymer” means that the composition comprises less than 1% by weight with respect to the total weight of the composition, preferably less than 0.5% by weight, preferably less than 0.3% by weight, preferably less than 0.1% by weight of (meth)acrylic polymer. Preferably, the composition is totally free from (meth)acrylic polymer. (Meth)acrylic polymer means a polymer comprising at least one acrylic acid monomer or at least one methacrylic acid monomer.

“Substantially free from silicone” means that the composition comprises less than 1% by weight with respect to the total weight of the composition, preferably less than 0.5% by weight, preferably less than 0.3% by weight, preferably less than 0.1% by weight of silicone. Preferably, the composition is totally free from silicone. Silicone means any silicone compound.

### Chitosan

The composition according to the invention comprises at least 0.01% by weight with respect to the total weight of the composition of at least one native chitosan having a molecular weight strictly greater than 3000 Dalton (3 kDa) (1 Da=1 g/mol).

The quantity of native chitosan is strictly less than 15% by weight with respect to the total weight of the composition.

Preferably, the native chitosan has a molecular weight greater than or equal to 10 kDa, preferably greater than or equal to 15 kDa, preferably greater than or equal to 20 kDa. Preferably, the native chitosan has a molecular weight between 10 kDa and 2 MDa, preferably between 15 kDa and 1.5 MDa, preferably between 20 kDa and 300 kDa, preferably between 20 kDa and 200 kDa.

Chitosan is not widely found in nature. It has only been reported in the exoskeletons of certain insects, such as termite queens, and in the cell walls of a particular class of fungi, namely zygomycetes.

Chitosan is obtained by deacetylation of chitin. Chitin is a polysaccharide composed of several N-acetyl-D-glucosamine units interlinked by a β-(1,4) type bond.

The ideal chemical structure of chitosan is a chain of β-D-glucosamine monomers linked by a glycoside (1→4) bond.

According to the invention, “chitosan” means any copolymer formed from N-acetyl-D-glucosamine and D-glucosamine constituent units, the degree of acetylation of which is less than 90%. Chitosan consists of glucosamine sugar units (deacetylated units) and N-acetyl-D-glucosamine units (acetylated units) interlinked by β (1,4) type bonds and represents a polymer of the poly(N-acetyl-D-glucosamine)-poly(D-glucosamine) type.

Preferably, the degree of acetylation of chitosan is less than or equal to 80%, preferably less than or equal to 70%, preferably less than or equal to 60%, preferably less than or equal to 50%, preferably less than or equal to 35%, preferably less than or equal to 25%, preferably less than or equal to 15%.

The degree of acetylation is the percentage of acetylated units relative to the total number of units, and can be determined by Fourier transform infrared spectroscopy (FTIR) or titration with a strong base.

The chitosan of the invention is preferably a polysaccharide prepared from a fungal source. It is in particular extracted and purified from safe and abundant food-grade or biotechnological fungal sources such as Agaricus bisporus or Aspergillus niger.

The chitosan of the invention is preferably derived from the mycelium of a fungus of the Ascomycete type, and in particular Aspergillus niger and/or a Basidiomycete fungus, in particular Lentinula edodes (shiitake) and/or Agaricus bisporus. Preferably, the fungus is Aspergillus niger.

The chitosan can be of GMO origin, but is preferably of non-GMO origin.

The chitosan according to the invention is native, i.e., it is not modified. In particular, it has not undergone any chemical modification.

One method for preparing chitosan is that described in the application WO03068824.

Preferably, the chitosan used in the invention is in powder form. It is in particular marketed by Kitozyme under the name Kiosmetine or Kionutrime.

The chitosan is preferably present in a quantity ranging from 0.01% to 14% by weight, preferably from 0.1% to 14% by weight, preferably from 0.1% to 12% by weight, preferably from 0.2% to 7% by weight, preferably from 0.25% to 5% by weight with respect to the total weight of the composition.

### Nanometric Surface-Treated Mineral Pigment

The composition according to the invention comprises at least one surface-treated mineral pigment, said pigment having a size less than or equal to 100 nm (“nanometric surface-treated mineral pigment”).

The term “pigments” means white or colored mineral particles, insoluble in an aqueous solution and intended for coloring the composition and/or the resulting deposit.

The treated pigments may be present, in the composition, at a content ranging from 1 to 70% by weight, preferably from 1 to 60% by weight, preferably from 1% to 50% by weight, preferably from 3% to 45% by weight, with respect to the weight of the composition, preferably from 4% to 30% by weight, preferably from 6% to 20% by weight, preferably from 6% to 15% by weight.

Preferably, the weight ratio between the nanometric surface-treated mineral pigment and chitosan (i.e., pigment:chitosan weight ratio) is greater than 1, preferably between 1 and 30. Preferably, it is between 1 and 25, preferably between 5 and 20.

### Mineral Pigments

“Mineral pigment” means any pigment that complies with the definition in Ullmann's Encyclopedia in the Inorganic Pigment chapter.

The size of the mineral pigment that can be used within the scope of the present invention is nanometric, i.e., less than or equal to 100 nm. According to a particular embodiment of the invention, the pigments have a size characterized by a D[50] less than 100 nm, preferably less than 50 nm, preferably between 5 and 40 nm, preferably between 10 and 30 nm.

The sizes are measured by measured by static light scattering by means of a commercial MasterSizer 3000® granulometer from Malvern, making it possible to apprehend the granulometric distribution of all the particles over a wide range that can range from 0.01 μm to 1000 μm. The data are processed on the basis of the classic Mie scattering theory. This theory is the most adapted for size distributions ranging from submicronic to multi-micronic, and makes it possible to determine an “effective” diameter of particles. This theory is described particularly in the book by Van de Hulst, H. C., “Light Scattering by Small Particles”, Chapters 9 and 10, Wiley, New York, 1957.

D[50] represents the maximum size of 50% of the particles by volume.

Any mineral pigment having a nanometric size can be used in the present invention.

Mention may be made, among the mineral pigments that can be used in the present invention, of titanium dioxide, iron oxides (black, yellow or red), zirconium or cerium oxides, zinc or chromium oxides, manganese violet, ultramarine blue, chromium hydrate and iron blue, metallic powders such as aluminum powder and copper powder.

Preferably, the mineral pigment is more particularly titanium dioxide.

Mention may also be made of nacres and/or pigments that have an optical effect that differs from a simple conventional hue effect (such as for example monochromatic pigments), once their size is nanometric.

The term “nacres” should be understood to mean iridescent or non-iridescent colored particles of any shape, which are in particular produced by certain mollusks in their shell or else are synthesized and which exhibit a color effect by optical interference.

The nacres may be selected from pearlescent pigments such as titanium mica coated with iron oxide, titanium mica coated with bismuth oxychloride, titanium mica coated with chromium oxide, titanium mica coated with an organic dye, and pearlescent pigments based on bismuth oxychloride. This may also involve mica particles at the surface whereof are superposed at least two successive layers of metal oxides and/or of organic dyes. By way of example of nacres, mention may also be made of natural mica coated with titanium oxide, with iron oxide, with natural pigment or with bismuth oxychloride. The nacres may more particularly possess a yellow, pink, red, bronze, orange, brown and/or copper color or glint. Among the pigments than can be used according to the invention, mention may also be made of those with an optical effect that differs from a simple conventional hue effect, i.e., a unified and stabilized effect such as that produced by conventional dyes, such as, for example, monochromatic pigments.

For the purpose of the invention, “stabilized” means absence of an effect of variability of color with the angle of observation or in response to a temperature change.

### Surface Treatment Agent

The surface treatment agent according to the invention is a hydrophobic agent. It is chosen preferably from fatty acids, metal salts and mixtures thereof.

### Fatty Acids

“Fatty acid” means a carboxylic acid with an aliphatic chain comprising 8 to 30 carbon atoms, preferably 12 to 24 carbon atoms and more preferably, 16 to 18 carbon atoms. The fatty acid may be liquid or non-liquid.

Liquid fatty acid means a fatty acid that is liquid at ordinary temperature (25° C.) and at atmospheric pressure (760 mm Hg, i.e., 1.013.105 Pa).

The fatty acids according to the invention may be saturated or unsaturated.

The fatty acids suitable for implementing the invention are more particularly chosen from linear or branched, saturated or unsaturated acids, including from 8 to 30 carbon atoms.

Preferably, the fatty acid is chosen from stearic acid, palmitic acid, palmitoleic acid, linoleic acid, caprylic acid, myristic acid, lauric acid, behenic acid and isostearic acid.

### Metal Salts

The metal salts are preferably hydroxides, for example aluminum hydroxides; hydroxides of alkali metals such as sodium or potassium; or hydroxides of alkaline earth metals such as magnesium.

Preferably, the metal salt is aluminum hydroxide.

Preferably, the surface treatment agent according to the invention is a mixture of fatty acid and a metal salt, preferably a mixture of stearic acid and aluminum hydroxide.

Preferably, the nanometric surface-treated mineral pigment according to the invention is nanometric titanium dioxide treated with aluminum hydroxide and stearic acid, such as that marketed under the name MICRO TITANIUM DIOXIDE MT-100 T V by TAYCA.

### Physiologically Acceptable Aqueous Medium

The composition according to the invention comprises a physiologically acceptable aqueous medium. Said medium comprises water.

The water used can be sterile demineralized water and/or floral water such as rose water, cornflower water, chamomile water or linden water, and/or a spring or natural mineral water.

The composition preferably comprises at least 5% by weight of water with respect to the total weight of the composition, preferably at least 10% by weight, preferably at least 20% by weight, preferably at least 30% by weight, preferably at least 40% by weight.

The composition preferably comprises from 5% to 95% by weight of water with respect to the total weight of the composition, more preferably from 10% to 85%, even more preferably from 20% to 80%, even more preferably from 25% to 70%, even more preferably from 28% to 60%, even more preferably from 30% to 50%.

The aqueous phase may also comprise at least one organic solvent miscible in water at 25° C.

Preferably, the water-miscible organic solvent is chosen from alcohols, polyols and mixtures thereof.

Among the alcohols, mention may be made of C₁-C₁₀, more preferably C₁-C₅, alcohols, such as ethanol, isopropanol, propanol and butanol.

The polyol is, preferably, chosen from polyols having from 2 to 20 carbon atoms, preferably from 2 to 6 carbon atoms, such as glycerol, diglycerol, propyleneglycol, isoprene glycol, dipropyleneglycol, butylene glycol, hexylene glycol, 1,2-propanediol, 1,3-propanediol, pentylene glycol, polyethyleneglycols having from 2 to 200 ethylene oxide units and mixtures thereof.

The composition may comprise from 0.1% to 25% by weight of water-miscible organic solvent, with respect to the total weight of the composition, more preferably from 0.5% to 20% by weight, even more preferably from 0.9% to 15% by weight.

### Alpha Hydroxy Acid (AHA)

The composition according to the invention may comprise at least one AHA.

According to the invention, alpha hydroxy acid means a carboxylic acid having at least one hydroxy function occupying an alpha position on said acid (carbon adjacent to a carboxylic acid function). This acid may be presented in the final composition in the form of free acid and/or in the form of one of its associated salts (salts with an organic base or an alkali in particular), in particular according to the final pH imposed on the composition.

The α-hydroxy acids (alpha hydroxy acids or AHA) are for example chosen from lactic acid, citric acid, methyllactic acid, glucuronic acid, glycolic acid, pyruvic acid, 2-hydroxy-butanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, 2-hydroxytetradecanoic acid, 2-hydroxy-hexadecanoic acid, 2-hydroxyoctadecanoic acid, 2-hydroxytetra-cosanoic acid, 2-hydroxyeicosanoic acid; mandelic acid; phenyllactic acid; gluconic acid; galacturonic acid; aleuritic acid; ribonic acid; tartronic acid; tartaric acid; malic acid; fumaric acid; salts thereof and mixtures thereof.

According to a preferred embodiment, the alpha hydroxy acid is chosen from lactic acid, citric acid, malic acid, tartaric acid and salts thereof. More particularly, the alpha hydroxy acid is chosen from lactic acid, citric acid, salts thereof and mixtures thereof.

The alpha hydroxy acid(s) may be present in a quantity ranging from 0.001 to 10% by weight, from 0.005 to 5% by weight, preferably from 0.01 to 3% by weight with respect to the total weight of the composition.

### Surfactants

The composition may comprise at least one surfactant. The surfactant is preferably chosen from among anionic, cationic, non-ionic, zwitterionic and amphoteric surfactants.

Preferably, it is chosen from among:
a) non-ionic surfactants, in particular of HLB greater than or equal to 8 to 25° C., used alone or in a mixture. Mention may be made in particular of esters of fatty acid and of polyglycerol, esters of fatty acid and of polyethylene glycol, alkylC8-C30 (poly)glycosides or mixtures thereof. In particular, the non-ionic surfactant may be an ester of fatty acid comprising 10 to 18 carbon atoms and of polyglycerol comprising 2 to 10 glycerol units. In a preferred embodiment of the invention, the fatty acid ester comprises 12 carbon atoms and 10 glycerol units, and is polyglyceryl monolaurate comprising 10 glycerol units, i.e., polyglyceryl-10 monolaurate (available under the trade name DERMOFEEL G 10 L from Dr Straetmans). It may also be polyglyceryl-2 monooleate (available under the trade name SUNSOFT Q-17D (G)-C from TAIYO KAGAKU), or polyglyceryl-4 monocaprate or PG-4 caprate (available under the trade name TEGOSOFT PC 41 from EVONIK GOLDSCHMIDT).
b) anionic surfactants such as:
   polyoxyethylenated fatty acid salts and particularly those derived from alkaline salts, and mixtures thereof;
   phosphoric esters and their salts such as “DEA oleth-10 phosphate” (Crodafos N 10N from CRODA) or monopotassium monocetyl phosphate (Amphisol K from Givaudan);
   sulfosuccinates such as “Disodium PEG-5 citrate lauryl sulfosuccinate” and “Disodium ricinoleamido MEA sulfosuccinate”;
   alkylethersulfates such as sodium lauryl ether sulfate;
   isethionates;
   acylglutamates such as “Disodium hydrogenated tallow glutamate” (AMISOFT HS-21 RR marketed by AJINOMOTO) and sodium stearoyl glutamate (AMISOFT HS-11 PF® marketed by AJINOMOTO) and mixtures thereof;
   derivatives of soybeans such as potassium soyate;
   citrates, such as Glyceryl stearate citrate (Axol C 62 Pellets from Degussa);
   derivatives of proline, such as Sodium palmitoyl proline (Sepicalm VG from Seppic), or the Mixture of Sodium palmitoyl sarcosinate, Magnesium palmitoyl glutamate, palmitic acid and Palmitoyl proline (Sepifeel One from Seppic);
   lactylates, such as Sodium stearoyl lactylate (Akoline SL from Karlshamns AB);
   sarcosinates, such as sodium palmitoyl sarcosinate (Nikkol sarcosinate PN) or the mixture of Stearoyl sarcosine and Myristoyl sarcosine 75/25 (Crodasin SM from Croda);
   sulfonates, such as Sodium C14-17 alkyl sec sulfonate (Hostapur SAS 60 from Clariant);
   glycinates, such as sodium cocoyl glycinate (Amilite GCS-12 from Ajinomoto);
   C16-C30 fatty acid salts in particular those derived from amines, such as triethanolamine stearate and/or amino-2-methyl-2-propane di-ol-1,3 stearate;
c) amphoteric surfactants, such as N-acyl-aminoacids such as N-alkyl-aminoacetates, disodium cocoamphodiacetate and amine oxides such as stearamine oxide or even silicone surfactants such as dimethicone copolyol phosphates such as that sold under the trade name PECOSIL PS 100® by PHOENIX CHEMICAL;
and mixtures thereof.

### pH of the Composition

The composition according to the invention has a pH less than or equal to 7, preferably less than or equal to 6.5, preferably less than or equal to 6.3. Advantageously, the pH of the composition is between 3 and 6.3, preferably between 4 and 6.3.

Preferably, the cosmetic composition according to the invention comprises at least one base and/or at least one acid. The base and the acid according to the invention are known and conventionally used in the cosmetics field.

The base and/or the acid are in particular used to adjust the final pH of the composition between 3 and 6.3.

The acid may be for example citric acid.

The base can be chosen from mineral bases such as for example alkali metal hydroxides, sodium hydroxide, potassium hydroxide.

Preferably, the base of the composition is an alkali metal hydroxide, preferably sodium hydroxide or potassium hydroxide.

The composition according to the invention may comprise at least one base at an active substance content ranging from 0.5% to 10% by weight, with respect to the total weight of the composition, particularly from 1% to 5% by weight, preferably ranging from 1% to 4% by weight.

The composition according to the invention can be obtained conventionally by those skilled in the art.

It also relates to a process for make-up and/or care of the skin and/or skin appendages, wherein the composition according to the invention is applied on the skin and/or skin appendages.

The following examples make it possible to understand the invention better, without being in any way limitative. The raw materials are referred to with the chemical or INCI name thereof. The quantities indicated are as a % by weight of raw materials with respect to the total weight of the composition (% w/w), unless specified otherwise.

### EXAMPLE 1: PREPARATION OF A COMPOSITION ACCORDING TO THE INVENTION AND COMPARISON WITH COMPARATIVE COMPOSITIONS

Composition A according to the invention and comparative composition B were prepared by mixing the ingredients from Table 1.

Composition A according to the invention contains TiO2 surface-treated with aluminum hydroxide and stearic acid.

Comparative composition B does not contain chitosan.

Composition A according to the invention has a good carbon footprint in particular by promoting the use of renewable raw materials and/or those with a good wilderness quality index and/or of natural origin, and more particularly those of plant origin.

The results are in Table 1.

TABLE 1

| | A | |
|---|---|---|
| | according | |
| Ingredients | to the | B |
| (% w/w) | invention | comparative |
| Chitosan | 1 | — |
| Lactic acid | 0.5 | 0.5 |
| Glycerol | 1 | 1 |
| Polyglyceryl-10 | 0.1 | 0.1 |
| laurate | | |
| Nanometric titanium | 15 | 15 |
| oxide (TiO2) pigment | | |
| treated with | | |
| aluminum hydroxide | | |
| and stearic acid | | |
| (MICRO TITANIUM | | |
| DIOXIDE MT-100 T | | |
| V from TAYCA) | | |
| Water | QS | QS |
| | 100 | 100 |

The formulas were manufactured according to the same process as follows: Lactic acid and chitosan were mixed with a rotor-stator from VMI until the chitosan had fully dissolved. Glycerol and polyglyceryl-10 laurate were added under gentle stirring. TiO2 was then added gradually and kept under stirring for 20 min.

### Evaluations:

### Macroscopic:

The samples were observed 24 h after manufacture to detect any phase separation of the systems.

### Resistance:

In order to evaluate the resistance to dry, water and oil friction, 30 μl of formula was deposited on 5×5 cm², on black plates. Each spreading was performed with a finger by performing 5 back-and-forth actions, followed by slight circles over the entire surface. The deposits are dried in the open air for 3 hours. They were then subjected to various frictional stresses:
Dry friction: each deposit was rubbed with a finger in 15 back-and-forth actions;
Water friction: each deposit was rubbed with a finger in 15 back-and-forth actions after water deposition;
Water friction+dry cloth friction: each deposit was rubbed around with a finger 15 times then wiped with a dry cloth;
Oil (OCTYLDODECANOL) friction+dry cloth friction: each deposit was rubbed around with a finger 15 times then wiped with a dry cloth.

### Results:

### Macroscopic:

The samples display no phase separation. However, chitosan allows better stabilization of the TiO2 dispersion.

### Resistance:

All of the deposit resistance evaluations subjected to various stresses show a clear difference between the samples with and without chitosan. The deposits with chitosan remain intact after stress, whereas the deposits without chitosan are substantially degraded. All of the results are compiled in the following table:

TABLE 2

| | | |
|---|---|---|
| Formula | | |
| Chitosan | A | B |
| Presence/Absence | Present | Absent |

Resistance score (from 1 (−) to 5 (+))

| | | |
|---|---|---|
| Dry friction | 4 | 2 |
| Water friction | 5 | 2 |
| Water + Dry Cloth | 5 | 2 |
| friction | | |
| Oil + Dry Cloth | 4 | 1 |
| friction | | |

Macroscopic evaluation

| | | |
|---|---|---|
| Appearance of | Homogeneous | Phase |
| storage jar after | | separation |
| 24 h | | |

Comparative formula without chitosan B, in addition to displaying a rapid phase separation, shows on the surface dewetted TiO2 which forms a potentially inhalable powder cloud.

### EXAMPLE 2: EVALUATION OF SPF OF A COMPOSITION ACCORDING TO THE INVENTION AND COMPARISON WITH A COMPARATIVE COMPOSITION

The idea consists of comparing the in vitro Sun Protection Factor (SPF) of an aqueous formula according to the invention versus a comparative oily formula, with the same TiO2 in the same quantity.

The two formulas tested were prepared as described in example 1.

TABLE 3

| | | Formula |
|---|---|---|
| | | according |
| | Comparative | to the |
| Ingredient | formula | invention |
| Water | | QS |
| | | 100 |
| Glycerol | | 1 |
| Nanometric | 15 | 15 |
| titanium oxide | | |
| (TiO2) pigment | | |
| treated | | |
| with aluminum | | |
| hydroxide and | | |
| stearic acid | | |
| (MICRO | | |
| TITANIUM | | |
| DIOXIDE MT- | | |
| 100 T V from | | |
| TAYCA) | | |
| ISONONYL | 83.5 | |
| ISONONANOATE | | |
| Polyhydroxystearic | 0.75 | |
| acid | | |
| POLYGLYCERYL- | | 0.1 |
| 10 LAURATE | | |
| POLYGLYCERYL-6 | 0.75 | |
| POLYRICINOLEATE | | |
| Lactic acid | | 0.5 |
| CHITOSAN | | 1 |

### In Vitro SPF Evaluation

### Filter Efficiency Evaluation Protocol

In-vitro: The sun protection factor (SPF) is determined according to the “in vitro” method described by M. Pissavini et al in International Journal of Cosmetic Science, 40, 263-268 (2018), based on the initial absorbance.

The in vitro UVA protection factor UVA (UVAPF) of a sun protection product against UVA radiation is calculated mathematically by in vitro spectral modeling according to the ISO 24443:2012 (Fr) protocol.

Each composition is applied on six rough PMMA plates, in the form of a homogeneous and regular deposit at a rate of 1 mg/cm². Each composition is spread using an automated robot which makes regular and uniform movements on three plates referred to as HD6 (molded granular plates) and three plates referred to as SB6 (sand-blasted granular plates). The plate is weighed before and after spreading. Once the six plates have been spread, they are left to stand in Thermo-Masters in the dark at 25° C. for 30 minutes. The measurements are made using a Labsphere UV-1000S spectrophotometer. Nine measurements per plate are made, then are analyzed using an Excel spreadsheet providing the SPF and UVAPF values of the measured composition.

TABLE 4

| | | Standard | | Standard |
|---|---|---|---|---|
| | In | deviation | | deviation |
| | vitro | (SPF | | (UVA |
| | SPF | DA) | UVAPF | DA) |
| Comparative | 46.3 | 3.9 | 8.3 | 0.5 |
| formula | | | | |
| Formula | 67.8 | 7 | 8.35 | 0.65 |
| according | | | | |
| to the | | | | |
| invention | | | | |

The results show a 46.4% gain in SPF in vitro with the formula according to the invention.

Conclusion: a composition according to the invention makes it possible to boost the SPF with respect to a comparative oily composition containing the same TiO2 in the same quantity.

### EXAMPLE 3: COMPARISON BETWEEN A COMPOSITION ACCORDING TO THE INVENTION AND A COMPARATIVE COMPOSITION COMPRISING A NANOMETRIC TIO2 WITHOUT SURFACE TREATMENT

The two formulas tested were prepared as described in example 1.

| INGREDIENTS (% W/W) | INVENTION | COMPARATIVE |
|---|---|---|
| WATER | Qs | Qs |
| | 100 | 100 |
| GLYCERIN | 1.00 | 1.00 |
| Nanometric titanium | 10.00* | — |
| oxide (TiO2) | | |
| pigment treated with | | |
| aluminum hydroxide | | |
| and stearic acid | | |
| (MICRO TITANIUM | | |
| DIOXIDE MT-100 | | |
| T V from TAYCA) | | |
| LACTIC ACID | 0.60 | 0.60 |
| HYDROXYPROPYL | 0.50 | 0.50 |
| GUAR | | |
| POLYGLYCERYL- | 0.10 | 0.10 |
| 10 LAURATE | | |
| CHITOSAN | 100 | 1.00 |
| Nanometric titanium | — | 10.00 |
| oxide (TiO2) | | |
| pigment without | | |
| surface treatment | | |
| (MT-700B from | | |
| Tayca, particles of | | |
| size less than 100 | | |
| nm ; 100% of | | |
| active material) | | |

*Due to surface treatment, this corresponds to 8.2% of active material (TiO2) by weight of the total weight of the composition

### Evaluations & Results:

Deposit resistance evaluations (dry friction, water+dry cloth friction and oil+dry cloth friction) were measured according to the same protocol as in example 1.

The results are as follows:

| | | |
|---|---|---|
| Formula | Invention | Comparative |

Resistance score (from 1 (−) to 5 (+))

| | | |
|---|---|---|
| Dry friction | 4 | 1 |
| Water + Dry Cloth | 4 | 1 |
| friction | | |
| Oil + Dry Cloth | 5 | 5 |
| friction | | |

Conclusion: with an amount of nano TiO2 by weight that is lower in the formula of the invention (8.2% by weight of nano TiO2) as compared to the comparative formula (10% by weight of nano TiO2), the dry and water friction is improved thanks to the surface treatment of TiO2 according to the invention.

The comparative formula does neither resist to dry friction nor to water friction.

### In Vitro SPF Evaluation

In vitro SPF was evaluated according to the same protocol as in example 2.

The results are as follows:

| | In | Standard | | Standard |
|---|---|---|---|---|
| | vitro | deviation | | deviation |
| | SPF | (SPF DA) | UVAPF | (UVA DA) |
| Invention | 346 | 0.5 | 5.6 | 0.2 |
| Comparative | 194 | 1.2 | 12.5 | 0.50 |

Conclusion: The results show a 78% gain in SPF in vitro with the formula according to the invention.

Comparative formula allows a higher filtration in UVAPF.

1. A cosmetic composition comprising, in a physiologically acceptable aqueous medium:
a) at least 0.01% by weight with respect to the total weight of the composition of native chitosan having a molecular weight strictly greater than 3000 Dalton, this quantity being strictly less than 15% by weight, and
b) at least one surface-treated mineral pigment, the pigment having a size less than or equal to 100 nm.

2. The composition according to claim 1, wherein the native chitosan has a molecular weight greater than or equal to 10 kDa.

3. The composition according to claim 1, wherein the native chitosan has a degree of acetylation less than or equal to 80%.

4. The composition according to claim 1, wherein the native chitosan is a polysaccharide prepared from a fungal source.

5. The composition according to claim 1, wherein the native chitosan is present in a quantity ranging from 0.01% to 14% by weight with respect to the total weight of the composition.

6. The composition according to claim 1, which comprises from 1% to 70% by weight with respect to the weight of the composition of treated mineral pigment(s).

7. The composition according to claim 1, wherein the mineral pigment is titanium dioxide.

8. The composition according to claim 1, wherein the mineral pigment has a size characterized by a D[50] less than 100 nm, where D[50] represents the maximum size of 50% of the particles by volume.

9. The composition according to claim 1, wherein the pigment is treated with a hydrophobic surface treatment agent.

10. The composition according to claim 1 preceding claims, wherein the surface-treated pigment is titanium oxide treated with aluminum hydroxide and stearic acid.

11. The composition according to claim 1, wherein the physiologically acceptable aqueous medium comprises at least 5% by weight of water with respect to the total weight of the composition; and optionally at least one organic solvent miscible in water at 25° C. chosen from alcohols, polyols and mixtures thereof.

12. The composition according to claim 1, which comprises at least one alpha hydroxy acid; salts thereof and mixtures thereof.

13. The composition according to claim 1, which has a pH less than or equal to 7.

14. The composition according to claim 1 which is substantially free from (meth)acrylic polymer and/or silicone.

15. The composition according to claim 1 wherein the weight ratio between the nanometric surface-treated mineral pigment and chitosan (i.e., pigment:chitosan weight ratio) is greater than 1.

16. A process for make-up and/or care of the skin and/or skin appendages, wherein the composition according to claim 1 is applied on the skin and/or skin appendages.

17. The composition according to claim 2, wherein the native chitosan has a degree of acetylation less than or equal to 80%.

18. The composition according to claim 2, wherein the native chitosan is a polysaccharide prepared from a fungal source.

19. The composition according to claim 3, wherein the native chitosan is a polysaccharide prepared from a fungal source.

20. The composition according to claim 2, wherein the native chitosan is present in a quantity ranging from 0.01% to 14% by weight with respect to the total weight of the composition.
